(19) United States
(12) Patent Application Publication
(10) Pub. No.: US 2025/0116651 A1
(43) Pub. Date: Apr. 10, 2025

(54) AUTOMATED VERIFICATION AND GUIDANCE FOR TEST PROCEDURES

(71) Applicant: ATOMO DIAGNOSTICS LIMITED, Leichhardt, New South Wales (AU)

(72) Inventors: Rohit Ketkar, Leichhardt (AU); Chandra Sukumar, Leichhardt (AU); John Kelly, Leichhardt (AU)

(73) Assignee: ATOMO DIAGNOSTICS LIMITED, Leichhardt, New South Wales (AU)

(21) Appl. No.: 18/688,330

(22) PCT Filed: Sep. 2, 2022
(86) PCT No.: PCT/AU2022/051076

§ 371 Date: Feb. 29, 2024

(30) Foreign Application Priority Data
Sep. 2, 2021 (AU) 2021902844

(51) Int. Cl. G01N 33/487; G06T 7/00; G16H 10/60

(52) U.S. Cl. CPC G01N 33/48792; G06T 7/0012; G06T 2207/20081; G16H 10/60

(57) ABSTRACT

A method and system are provided to allow for the verification that a test, for example a point of care blood test, has been correctly performed. A software application on an imaging device, such as a smartphone, captures specific images at one or more stages of the test procedure, which are processed and analysed to ensure that they correspond to the correct status of the test unit at that stage in the procedure.

### TECHNICAL FIELD

The present invention relates to test procedures carried out on user operated test devices, such as point of care and self-test devices, for example lateral flow or other rapid test devices.

### BACKGROUND OF THE INVENTION

Testing for various indications using single use, relatively inexpensive devices is a growing part of medical practice, as well as to other fields of activity. These may be, for example, lateral flow or other rapid test devices, intended for point of care, professional or at home testing. The tests may be for use with samples such as blood, saliva, mucus, urine. Typical tests include for specific infective agents or antibodies, metabolites, specific molecules, or combinations of these.

Such tests generally involve a series of actions which the user is required to undertake. In the case of a blood test, for example, the steps may include lancing a finger to obtain a suitable drop of blood, placing the blood in a specific location on a device, operating the device to deliver the sample to a location, and releasing buffer or a reagent into a specific location. Some of these steps may be manually carried out by the user using a kit of parts supplied with the device, or may be effected using mechanical or electronic components in the test device.

It is known to use imaging to determine or at least indicate a result from such tests. For example, a lateral flow test will generally have a test line, which if present indicates a positive test for the respective attribute, and a control line that indicates that a valid test has occurred. Imaging can be used to read these lines, apply appropriate software processing to determined (for example) that a sufficient intensity relative to some reference has been reached, and thereby indicate the outcome. Some commercially available disposable devices, for example the Clearblue Digital Pregnancy Indicator device, use on-board electronics to assess the control and test line in a lateral flow type device, and provide an indication of the result—in this case, whether the urine tested indicates that the user is pregnant.

U.S. Pat. No. 9,903,857 to Polwart et al discloses the use of camera in a portable device, such as a mobile phone, in order to image a testing apparatus and determine a test result, for example using the intensity of test and control lines.

British patent application GB2569803 by Novarum DX LTD discloses the use of an imaging frame in a process for imaging a test structure, in order to determine a test outcome value by analysis of the target measurement region.

However, the accuracy of the test outcome, whether electronically or visually determined, is highly dependent upon whether the correct procedures have been followed by the user, relative to the correct test protocol. The test result is not reliable unless each step has been carried out by the user in a correct manner, and in the correct order.

It is an object of the present invention to facilitate automated assistance and/or verification for carrying out a test procedure with a user operated test device.

### SUMMARY OF THE INVENTION

In a first broad form, the present invention provides a method using imaging to confirm that at least a part of a test procedure has been correctly performed. In another broad form, the present invention provides a method using imaging to guide user in the correct performance of a step, or to confirm that a user has correctly performed an intermediate step in a procedure.

According to one aspect, the present invention provides a method for verifying the correct operation of a procedure on a test unit, using an imaging device, including the steps of:
(a) providing a software application on the imaging device which is adapted to guide a test process using the test unit, and to capture images from the imaging device for processing;
(b) at one or more stages in the test process, the software application directing the user to capture an image of the test unit;
(c) processing a thereby captured image so as to identify one or more predetermined features of the test unit;
(d) analysing the identified features to determine whether they meet determined requirements, and thereby determining whether the requirements have been met; and
(e) providing an indication that the procedure is verified or not verified, responsive to the determination.

According to another aspect, the present invention provides a test verification system, including a test unit, and an associated software application adapted to be loaded on an imaging device, wherein the software application is adapted to guide a test process which is to be carried out by a user using the test unit, and to capture images from the imaging device for processing;

wherein at one or more stages in the test process, the software application is adapted to direct the user to capture an image of the test unit, process the captured image so as to identify one or more predetermined features of the test unit; analyse the identified features to determine whether they meet determined requirements, and thereby determining whether the requirements have been met; and to provide an indication to the user that the procedure is verified or not verified, responsive to the determination.

Other aspects of the present invention include a stored software application adapted to operatively carry out the method, and a test unit including a link or code to connect to and associate with a stored software application.

Implementations of the invention allow for a verification that at least some of the correct procedure for operating at test unit has been complied with, based on the correct positioning of the device and samples.

### BRIEF DESCRIPTION OF THE DRAWINGS

Illustrative embodiments of the present invention will be described in connection with the accompanying figures, in which:

FIGS. 1A to 1D are examples of test devices after the test procedure is completed;

FIGS. 1E and 1F are examples of alternative test devices;

FIG. 2A is a flowchart explaining the steps in an illustrative app to support test process verification;

FIG. 2B is a flowchart of an alternative process to FIG. 2A;

FIG. 3 is a software flowchart illustrating one software method for implementing the process of FIG. 2A;

FIGS. 4A and 4B illustrates a process of alignment of a guide to assist correct creation of an image as part of the implementation of the present invention;

FIGS. 5A to 5C illustrate partial and complete filling of a blood delivery tube;

FIG. 6A is a flowchart explaining the steps in an illustrative app to support with the tube filling process;

FIG. 6B is a software flowchart illustrating a software process to implement the process of FIG. 6A.

FIG. 7A illustrates the use of an alignment card; and

FIG. 7B illustrates an alignment card; and

FIG. 8 is a flowchart relating to a training process for one AI implementation.

### DETAILED DESCRIPTION

The present invention will be described with reference to a particular example of a testing device, using a lateral flow type blood test. However, the general principle of the present invention is applicable to a wide variety of test types, both for medical and other purposes.

The present invention may be applied, for example to tests such as lateral flow biochemical and immunological tests; chemical reagent tests; or any other type of user conducted test. The tests may be medical or for other purposes, for example veterinary, agricultural, environmental or other purposes. The test result may be intended to be read using a conventional visual inspection, for example test and control lines; using optical systems (whether on board or external such as a smartphone) for determining the test outcome; or using electrochemical or other systems to determine the result. The present invention should not be interpreted as limited to any specific type test or the objective of those tests.

In the context of medical tests, the present invention could be applied to any kind of sample testing which can be assessed using the kind of user operated devices discussed, for example testing of blood (including serum, plasma), urine, sputum, mucus, saliva or other body samples. It is noted that while the discussion is primarily in the context of a user operated test, the present invention is also applicable to tests intended for a professional, laboratory or hospital environment.

It will be appreciated that a fluid such as a buffer or reagent may be added to the test unit after the sample is delivered, or at the same time. In some applications, the sample may be pre-mixed with a buffer, reagent or other fluid prior to the mixed sample being delivered to the test device.

The present invention will generally be implemented in conjunction with a portable electronic device, for example a tablet or smartphone, with a suitable application (app) loaded in a conventional way. For example, a customer may purchase a test for home use, which includes a QR code linking to a website at which the appropriate app can be downloaded in the manner appropriate for the particular device, for example at an app store for an Android® or Apple® device. However, the present invention could be implemented using conventional PCs or other devices and systems if desired. The term imaging device is intended to be understood broadly as including personal computers, smartphones and other camera phones, tablets, and any other device adapted to take digital images.

In a preferred implementation, the QR or other code is associated with the specific test unit, so that it is identified and can be specifically link the test verification to a particular test unit on a particular day, and potentially thereby linked to the medical records for a specific user or patient. It may also allow a more generally directed app to select the specific steps, frames and guidance appropriate for that specific test unit.

It will be appreciated that there are devices and systems, for example as described in the prior art references above, which disclose the necessary systems to optically (or otherwise) read a specific test result. The present invention can be principle be implemented in conjunction with any result determining system. For example, the present invention could be implemented in conjunction with a device that itself automatically determines a test outcome, not associated with the app or functionality in the portable device. In other implementations, the result determining process could form part of the process in the portable device/app, and the same or related images could be used to determine both the test outcome, and to verify, guide or confirm that the correct protocol has been followed.

The present implementation will be described with particular reference to the Galileo system test device, https://atomodiagnostics.com/galileo-rdt-platform/. However, it will understood that this is merely one illustrative example of a test unit with which the present invention may be employed.

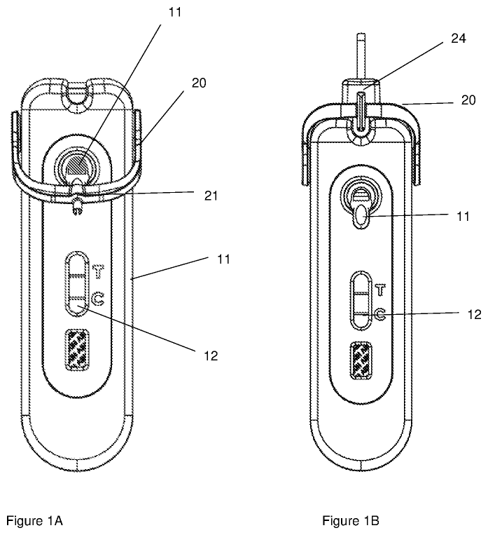

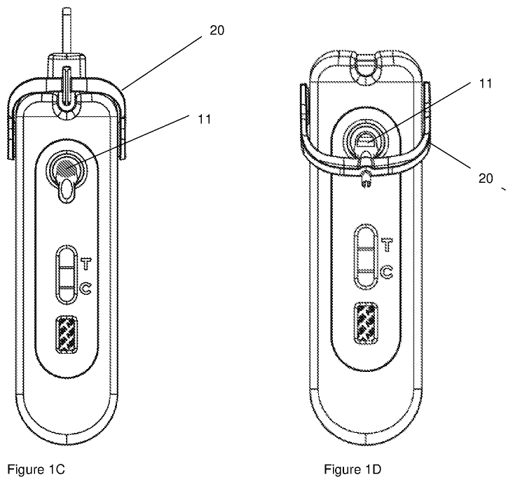

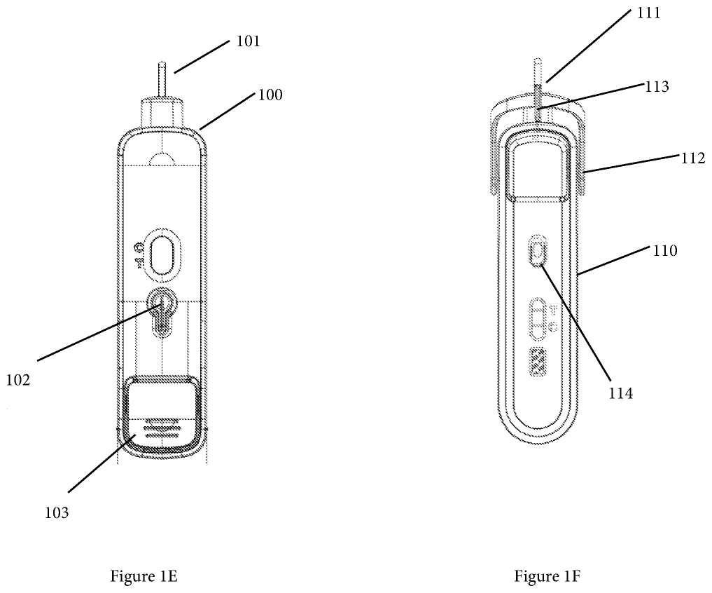

Considering FIG. 1A, the testing device in this example includes a body 10, a blood collection unit (BCU) 20, and a lancet 24. The body (in use) includes a lateral flow test strip, parts of which can be seen through the sample port 11 and the result window 12. BCU 20 includes a blood collection tube 21.

The general operation of the device is as follows. The user (who may be a person self testing, or a medical professional or other person assisting) first cleans, for example with a disinfecting wipe, the intended lancing site, typically a finger. The lancet 24 has a spring loaded release, which lances the finger (or other site) and then retracts to a non-operative position. A blood droplet is then expressed by the user.

The device is then positioned so that blood collection tube 21 engages the blood droplet, which is taken up into the blood collection tube 21 by capillary action. It is important that the tube is fully filled, so that the correct sample size is taken.

The BCU 20 is then rotated, to move to the delivery position, in which the blood collection tube 21 engages the test strip via the sample port 11. Contact allows blood to flow from the blood collection tube 21 onto the test strip.

The user then applies a reagent solution through the sample port 11. The lateral flow device then operates conventionally, to produce (for example) a control line and a test line in the result window.

It is axiomatic that the procedure needs to be followed correctly in order to obtain a valid result. If the procedure is not correctly carried out in a material way, then regardless of the status of the test and control lines, no valid result can be produced.

FIG. 1A shows a device where there is blood visible in the sample port 11, and the BCU 20 has been moved to the correct position. This is consistent with a correctly performed test.

However, in FIG. 1B, the BCU 20 has not been moved to the delivery position, and there is no blood visible in the sample port 11. This is not a valid test. In FIG. 1C, blood is visible in the sample port 11, but the BCU has not been moved to the delivery position. This is also not a valid test. In FIG. 1D, the BCU has been moved to the delivery position, but no blood is visible in the sample port 11.

FIG. 1E illustrates an alternative test device 100. This includes a lancet 101, a sample port 102, and an actuator 103. After correct operation in this case, blood should be visible in the sample port, the actuator 103 should be depressed (to bring the sample and fluid into contact with the test strip), and the lancet should have moved from the ready to the retracted position. FIG. 1F illustrates a further alternative device 110, including a lancet, BCU 112, blood collection tube 113, and sample port 114. After correct operation, BCU 113 should be moved from the rest position shown to a position where the blood collection tube 113 has engaged with the sample port 114. Blood should be visible in sample port 114, and the lancet 111 should have been fired and then retracted.

This implementation of the present invention provides an app, for example for a smartphone, in which a user is guided through the process, and as a result of images taken with the smartphone camera, the correct final status of the device can be verified, and hence provide an indication that the test result (whatever that is) is the result of a correctly performed process.

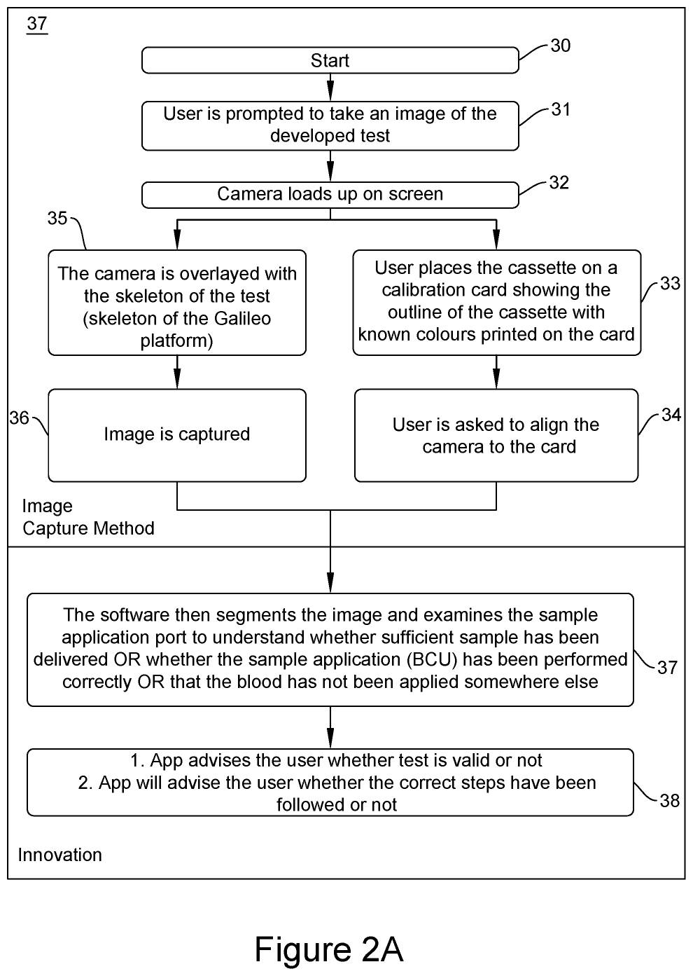

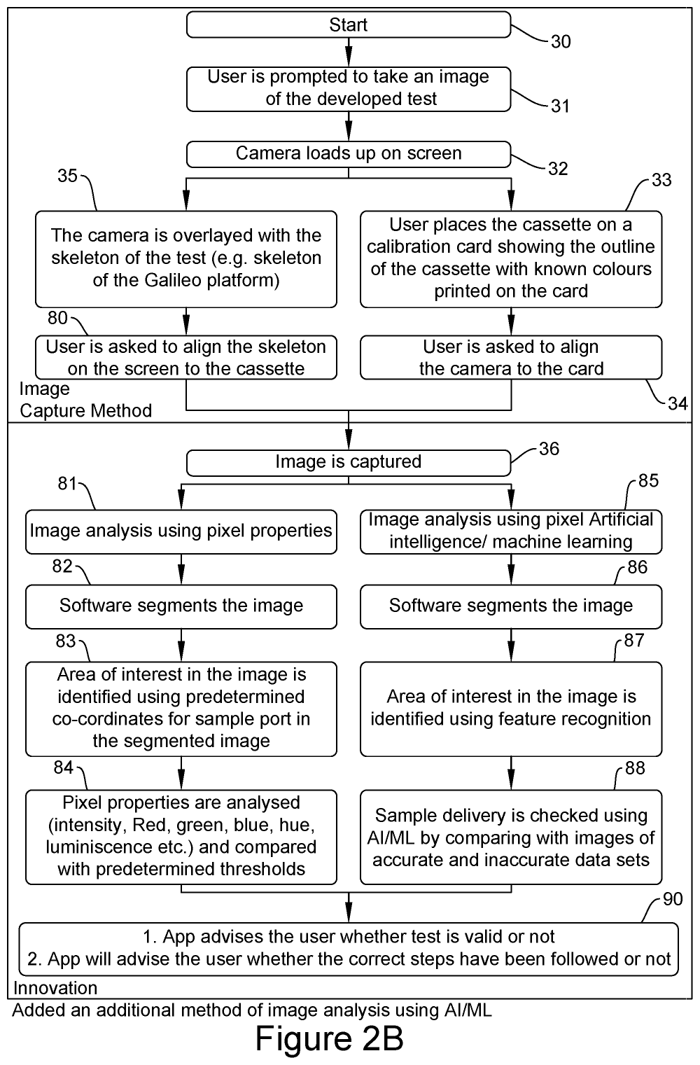

FIG. 2A is a flowchart which illustrates the steps required to be taken by the user according to one implementation.

At step 30, the start of the verification stage starts. The app prompts 31 the user to take an image of the test, at a stage when it is completed. This could be while the waiting time is still running, as part of the process for determining the test result itself, or a separate process undertaken shortly before or after the test result image is captured.

At this stage, the app opens the camera function and the camera viewer loads up on screen. In one implementation, the user is provided with a paper calibration device which includes the outline of the device, and potentially other visual features to assist the image processing software to align and correctly orient the image captured. The card preferably includes sections of one or more known colours, to provide a reference for the image processing software.

At 34, the app prompts the user to position the test device on a certain position on the cards, and align the camera in a particular way, for example as a plan view, relative to the test device. Once that is correct, the image may be captured, either by the user triggering the camera function, or by the software recognising that there is sufficient alignment and taking the image.

In an alternative implementation, the app includes an augmented reality function, in which a skeleton or outline (or other guiding image) of the test device is overlaid on the camera viewing screen 35. This guides the user to try and align the camera to correspond to the virtual image projected on the viewing screen. Once sufficient alignment is present, the camera may be manually triggered by the user, or automatically by the app 36.

In either case, once sufficient alignment is detected by the app, in a preferred form a change in colour or similar visual indication is provided to confirm this to the user.

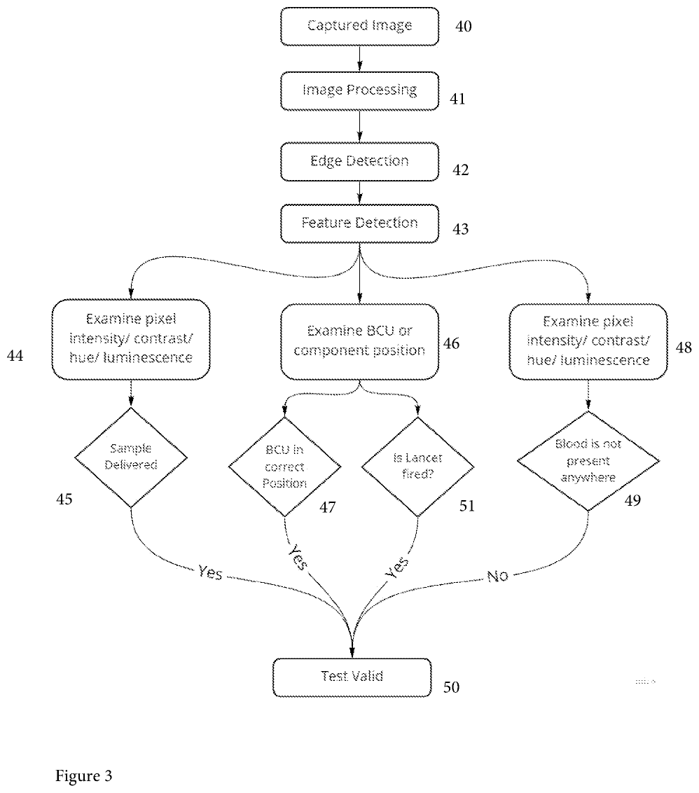

At step 37, as will be explained in more detail in relation to FIG. 3, a software algorithm within the app (preferably locally provided, or alternatively available via a cloud connection) segments the image and determines the status of specific features in the image relative to a status corresponding to a correct procedure.

At step 38, the app notifies (for example via a display) whether or not the test procedure is valid, and optionally (as discussed above) also delivers the test result after image analysis of the test and control lines (in this example). The app may also advise a central authority or local system, such as a system in a physician's office, of the result of the test. This may be loaded into a electronic patient record, either local or centralised, so as to record that a valid test procedure was undertaken.

FIG. 2B is a flowchart of an alternative process to that shown in FIG. 2A. The upper section, dealing with image capture, is largely the same. At step 35, an overlay of the skeleton or outline of the test device is generated on the screen, and at step 80, the app requests the user to align the skeleton to the image of the cassette (test unit) and the skeleton. The image is then captured at step 36.

The flowchart then has alternative pathways. On the left, at 81 image analysis using pixel properties is undertaken, and at 82 the image is segmented to identify areas with different characteristics.

At 83, the area of interest in the image (which is known in advance) is identified using the predetermined co-ordinates for the sample port, which are defined relative to the aligned skeleton. The area can then be identified in the segmented image.

At 84, pixel properties are analysed for the region of interest. These may include intensity, colour (red, green, blue), and luminescence, and compared with predetermined thresholds. These are selected according to the specific factor being verified, so for example to distinguish between a section of test which has received blood and an unaltered test.

This enables a determination of whether the step has been completed, and the app can correspondingly advise the user whether the step has been correctly completed.

The right hand pathway starts at step 85 with image analysis using pixels, but using artificial intelligence/machine learning tools. At step 86, the image is segmented, and at step 87, areas of interest in the image are identified using features recognition.

At 88, the correct delivery of the sample is verified using the AI/ML tools, comparing the identified area in the image with existing images of accurate and inaccurate tests. The result is then advised to the user in step 90.

FIG. 3 is a functional flow chart illustrating one software implementation of the present example. It will be appreciated that various approaches to image analysis could be used to determine the status of the salient features of the test unit, and that this is only one possible implementation. It will also be understood that this is specific to identifying the specific requirements for this particular example of a test, and for alternative tests different aspects may need to be checked, or checked in different ways.

The image is captured at 40 and the image processing function is commenced at step 41. The first function is edge detection 42, so that the edges of the test unit are identified. Once the edges have been identified (noting that the shape is known), the specific features of interest can be identified at 43.

In this example, there are three aspects that are important. First, whether it appears that blood has been delivered to the sample port. If no blood is present, then the test cannot be valid. At step 44, the pixels corresponding to the sample port are examined, to determine whether blood is present. This feature should have an irregular red area, compared to an unused sample port which will be uncoloured. This may be determined by, for example, colour, contrast, intensity, or a combination of these. At 45, a determination is made automatically whether a sample was delivered to the sample port.

The second aspect is determining whether the BCU 20 has been moved from the initial position to the delivery position. If it remains unmoved, or even is in some other position, then it is likely that the correct amount of blood has not be delivered, even If the indication at 45 is positive.

The software compares the detected BCU position relative to the body of the test unit with the known correct position at 46, and determines at 47 whether the BCU is in the correct position.

Similarly, the third aspect is to determine whether the lancet has been fired. In this device, the lancet retracts and has a different appearance after it has been operated to the ready state. The lancet position is determined at 46, and a determination is made at 51 as to whether the image is consistent with the lancet being fired.

The fourth aspect is to determine if there appears to be blood present at any other location, for example in the test/control window 12. This would indicate that the user has misunderstood the process and put their blood sample in the wrong place, and even if the other indications are positive, this will still indicate an invalid test.

At 48, the software examines the pixels away from the sample port, in order to determine if their characteristics are consistent with blood being present. As the edges are known for the test unit, and the sample port location is known, pixels corresponding to blood at other locations indicate incorrect operation. This could be limited to positions, for example the test/control window, where blood will definitely invalidate the test, but none the less there may appear to be a well developed test and control line. The result is determined at 49.

At step 50, if all 4 conditions are met, the test is determined to be valid. Otherwise, it is determined to be invalid.

Edge detection is a common algorithm used in image recognition software by comparing pixel properties with the neighbouring pixels. The first step in edge detection is noise reduction. This may conveniently be implemented by a filter, such as Gaussian blur.

The next step is to calculate pixel value intensity gradient, for example using an algorithms such as Sobel Kernel. It calculates the pixel value gradient and intensity.

A suitable edge detection algorithm can then be applied. For example, this could be Sobel edge detection, Canny edge detection, Prewitt edge detection or Roberts edge detection.

Image segmentation is the process of identifying and dividing the image into different types of areas. This is a well established technique in image processing.

Image segmentation can take place by utilising tools such as
i. Threshold—Where pixels below a certain value (intensity, RGB, brightness, contrast etc.) are differentiated from others.
ii. Graphical segmentation—In this technique, regions are segmented based on their position in the image.
iii. Clustering-Clustering algorithms such as K-means clustering are utilised to perform segmentation.
iv. Semantic segmentation—this technique uses deep learning to tag each region in the image. This technique requires training data set to be provided with predefined tags.

The present implementation preferably uses graphical segmentation, since the present implementation of the invention controls how the user captures the image by either loading a skeleton of the device on the screen or asking the user to use a stencil. We can definitively say which part of the image will be our region of interest.

If the artificial intelligence (AI)/machine learning (ML) approach is utilised, it will again be understood that there are many possible approaches to this task. One approach is to use a deep learning method, such as CNN (convoluted neural networks). CNN is a Deep Learning algorithm which can take in an input image, assign importance (learnable weights and biases) to various aspects/objects in the image and thereby differentiate one image from another.

The architecture is similar to connectivity pattern of neurons in the brain and was inspired by the organization of the visual cortex. CNN is composed of an input layer, an output layer, and many hidden layers in between. FIG. 9 illustrates the scheme of such a model.

A typical structure of hidden layers are as follows:
1. Convolution-puts the input images through a set of convolutional filters, each of which activates certain features from the images.
2. Rectified linear unit (ReLU) allows for faster and more effective training by mapping negative values to zero and maintaining positive values. This is sometimes referred to as activation, because only the activated features are carried forward into the next layer.
3. Pooling simplifies the output by performing nonlinear downsampling, reducing the number of parameters that the network needs to learn.

These operations are repeated many thousands of times to create a model. A typical training set for such networks is of the order of 5000 images.

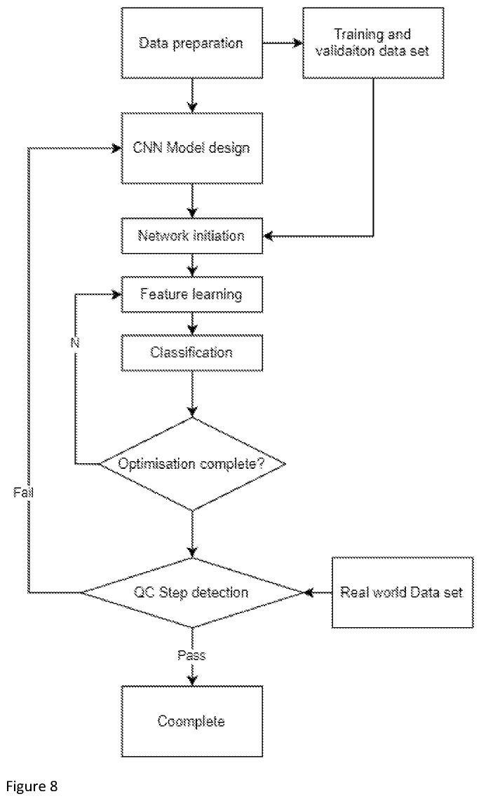

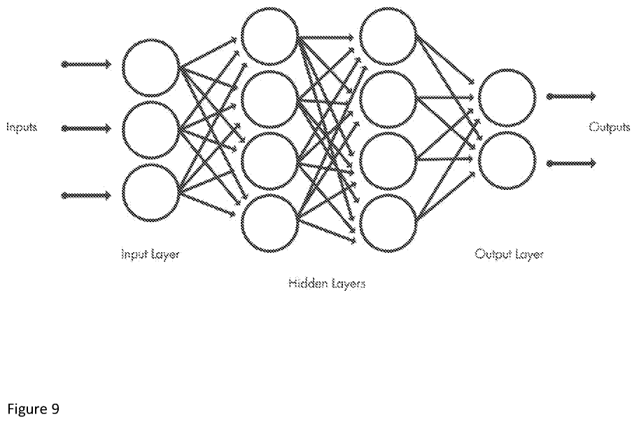

FIG. 8 illustrates a typical training workflow for a CNN model. It will be appreciated that there are many alternative AI models which could be used to provide the level of image discrimination required to implement the present invention.

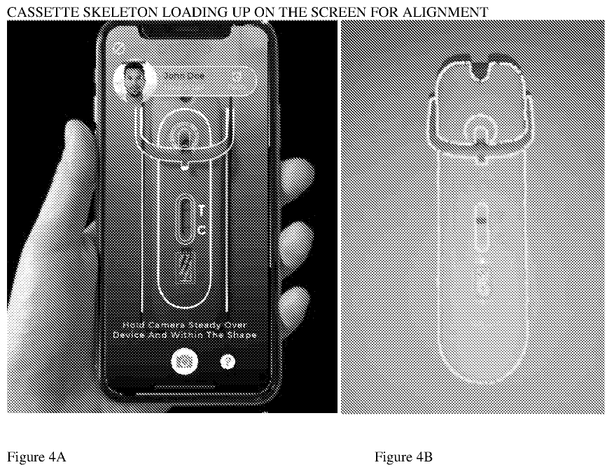

FIG. 4A illustrates the augmented image or skeleton which can be used according to one aspect to correctly align the test unit for imaging. This screen shows the virtual outline or augmented reality frame on the screen of the smartphone, for the user to align with the actual camera image on the phone. Once the outline is correctly aligned with an image from the smartphone camera, the outline turns yellow, to indicate correct positioning. This may then automatically take an image, or alternatively, be a guide to the user to take the image at that position.

The photo shows an outline or skeleton of the test unit displayed on the screen of a smartphone. FIG. 4B illustrates a image of the test unit, as visible as a virtual or on the smartphone screen,

It will be appreciated that the software functions of the present invention may be carried out in processing terms in a variety of ways. For example, part of the processing may be carried out locally on the smartphone or similar device, and part in a server or other remote system or cloud service contacted by the software application as part of its operation. In other implementations, the entire processing could occur in a local imaging device. In another implementation, the local device may be a thin client and simply provide an interface and capture images, the remainder of processing happening at a remote server or in a cloud service.

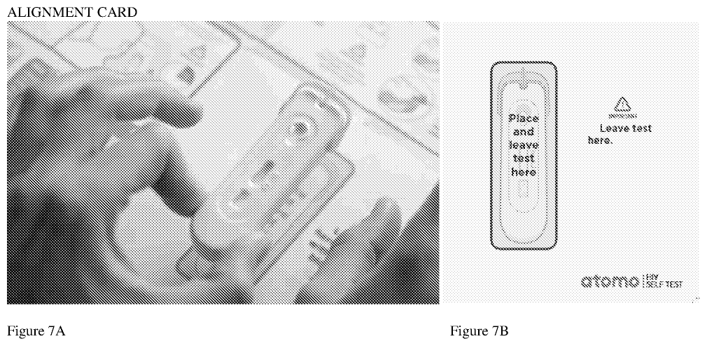

In an alternative implementation, a test card such as shown in FIG. 7B is provide with the kit. This is simply a piece of card or paper with an outline, to guide the user as to exactly where to place the test unit. FIG. 7A is a photo showing a user placing the test unit within the outline on the test card. The test card provides cues to assist with image alignment and assessment of the camera image, so that the software is provided with alignment and positioning information.

While the example described is focussed on determining whether the final status of a test device appear correct and consistent with a test protocol, the present invention may equally be applied to intermediate stages of the test, to verify that the user has performed them correctly, and in some cases to then allow the user to be guided to correct their error.

In the test device used in the example, the blood collection tube 21 on the blood collection unit is adapted to take up a specific volume of blood. For some tests, it is critical that the correct blood volume is used. If the tube is not fully filled, the volume will not be sufficient.

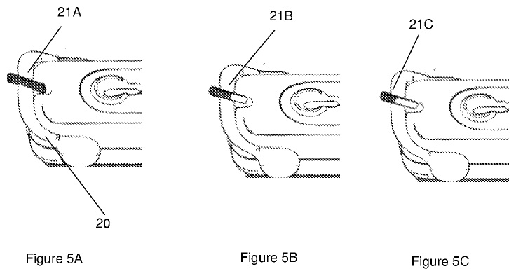

Referring to FIG. 5A, this shows a blood collection tube 21 which is full. However, FIG. 5B shows a blood collection tube with very little blood, and FIG. 5C shows a blood collection tube which is mostly filled but not completely. A procedure similar to that described in relation to FIG. 2 can be used to verify that the blood collection tube is correctly filled, and if not, in one form guide the user to collect additional blood. In another form, this is simply another condition used as part of the final validity determination.

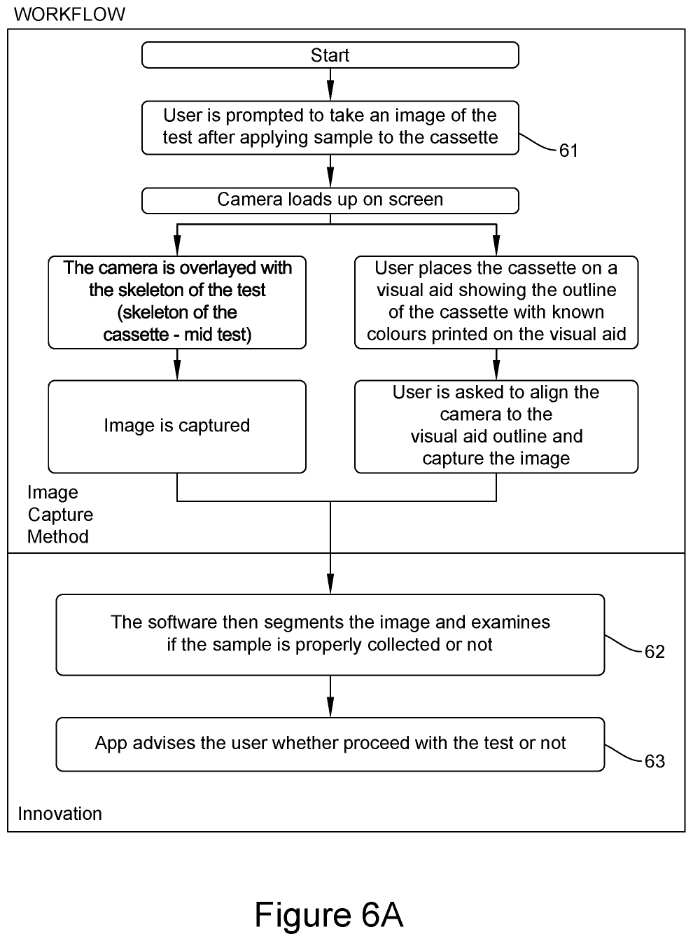

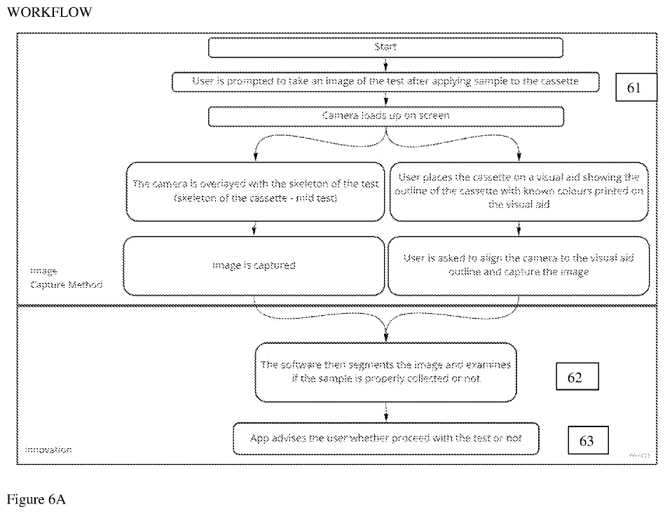

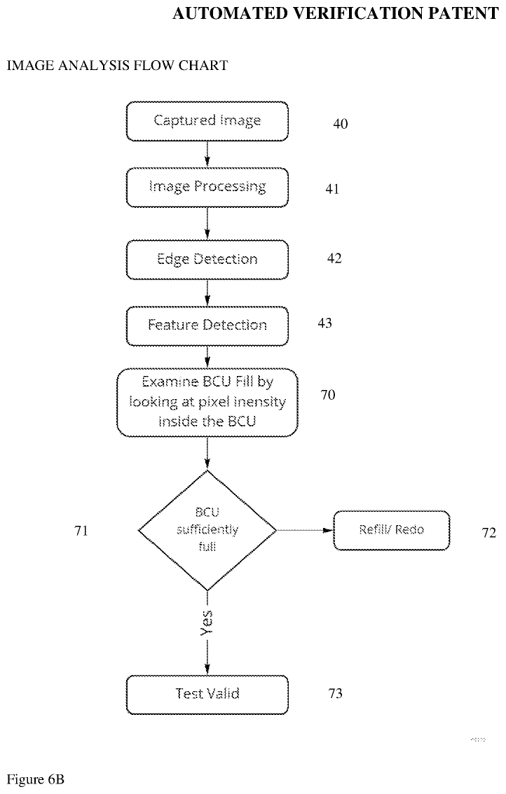

Referring to FIG. 6A, this shows a workflow very similar to FIG. 2, but modified as required for the specific intermediate testing to determine correct filling of the blood collection tube 21. After starting, at step 61 the user is prompted to take an image of the test after the blood has been taken up into tube 21. The image capture procedure is just as in FIG. 2. At step 62, the image is segmented, and examines the segment corresponding to the blood collection tube to see if the sample has been correctly collected. At step 63, the user is advised how to proceed, based upon the correct collection of the sample, or otherwise.

FIG. 6B explains the operation of the software for processing the captured image. Again, this process is similar in the early stages to that illustrated in FIG. 3. The image is captured at 40 and the image processing function is commenced at step 41. The first function is edge detection 42, so that the edges of the test unit are identified. Once the edges have been identified (noting that the shape is known), the specific features of interest can be identified at 43.

In this case, the feature of interest is the BCU. At 70, BCU fill is determined by looking at the pixel intensity inside the structure identified as the BCU. If the BCU is sufficiently full at 71, then the test, or at least this specific step, is determined to be valid and complete. If the determination at 71 is no, then the user is prompted to take an action, for example to take up additional blood into the blood collection tube.

It will be understood that the present invention may be broadly applied to verify whether any number of steps or procedures have been correctly carried out, whether in a point of care situation, home use, or a laboratory or other professional setting. The image capture and processing aspects of various implementations of the present invention may be used to provide an form of verification of tests and procedures.

For example, in the blood test context discussed above, the present invention may be applied to verify:
1. Whether blood/sample is present in the sample application region
2. Whether blood collection unit or other test component is in its rotated/intended position
3. Whether buffer has been added to the test (presence of control line)
4. To confirm whether the blood/sample is present in any other region of the test device
5. Whether the lancet has been fired or not.

While the example described are primarily with reference to blood, it will be appreciated that the principles are equally applicable to samples such as saliva, mucus, urine, or to samples mixed with a buffer or other liquid, for example post nasal swabs for COVID-19 or other respiratory conditions.

In that case, the system may be adjusted appropriately to allow for detection of less strongly coloured samples. For example, a buffer may be used with a coloured component to facilitate detection, a colour change could occur in the test strip in response to the deposit of a sample, or a specific colour or colour combination may be selected using image processing within the camera device, specific to the intended sample (or its interaction with the test strip).

It will be understood that in some implementations, the present invention may be applied to verify one or more intermediate steps in a test process, or to provide confirmation or even guidance to rectify incomplete or incorrect stages. It may be employed to verify one or more aspects of the final stage of a test process. In other implementations, both intermediate stages and final outcomes may be verified.

1. A method for verifying a correct operation of a procedure on a test unit, using an imaging device, including the steps of:
(a) providing a software application on the imaging device which is adapted to guide a test process using the test unit, and to capture images from the imaging device for processing;
(b) at one or more stages in the test process, the software application directing the user to capture an image of the test unit;
(c) processing a thereby captured image so as to identify one or more predetermined features of the test unit;
(d) analysing the identified features to determine whether they meet determined requirements, and thereby determining whether the requirements have been met; and
(e) providing an indication that the procedure is verified or not verified, responsive to the determination.

2. A method according to claim 1, wherein the identified features include one or more of the presence of a sample in a correct position on the test unit, the presence of a sample in an incorrect position on the test unit, the position of a movable component of the test unit, or whether a sample of the correct dimensions is present in a component of the test unit.

3. A method according to claim 1, wherein the method further includes communicating the indication to another device, so as to form part of a record of the test or a patient record.

4. A method according to claim 2, wherein the indication of the presence or absence of a sample uses one or more of the colour, contrast or intensity of the corresponding position on the test unit.

5. A method according to claim 1, wherein the imaging device further includes a display, the display showing a of the current view being imaged by the device, and a frame is generated within the display corresponding to at least part of the test unit, so as to guide the user to capture a correct image of the test unit.

6. A method according to claim 1, wherein the processing and/or analysing step uses a machine learning or other AI technique to determine whether the image corresponds to a correctly performed feature of the test.

7. A method according to claim 2, wherein the test unit is adapted to test a sample from medical purposes, the sample being selected from one or more of blood, saliva, sputum, mucus, serum, plasma, or other bodily fluids.

8. A method according to claim 1, wherein the software application and imagine device are adapted to communicate with a remote system, in order to facilitate one or more of the steps.

9. A test verification system, including a test unit, and an associated software application adapted to be loaded on an imaging device, wherein the software application is adapted to guide a test process which is to be carried out by a user using the test unit, and to capture images from the imaging device for processing; wherein at one or more stages in the test process, the software application is adapted to direct the user to capture an image of the test unit, process the captured image so as to identify one or more predetermined features of the test unit; analyse the identified features to determine whether they meet determined predetermined requirements, and thereby determining whether the requirements have been met; and to provide an indication to the user that the procedure is verified or not verified, responsive to the determination.

10. A system according to claim 9, wherein the identified features include one or more of the presence of a sample in a correct position on the test unit, the presence of a sample in an incorrect position on the test unit, the position of a movable component of the test unit, or whether a sample of the correct dimensions is present in a component of the test unit.

11. A system according to claim 10, wherein the software application is further adapted to communicating the indication to another device, so as to form part of a record of the test or a patient record.

12. A system according to claim 10, wherein the indication of the presence or absence of a sample uses one or more of the colour, contrast or intensity of the corresponding position on the test unit.

13. A system according to claim 9, wherein the imaging device includes a display, the display showing the current view being imaged by the device, and the software application is adapted to generate a frame within the display corresponding to at least part of the test unit, so as to guide the user to capture a correct image of the test unit.

14. A system according to claim 9, wherein the processing and/or analysis uses a machine learning or other AI technique to determine whether the image corresponds to a correctly performed feature of the test.

15. A system according to claim 9, wherein the software application and imagine imaging device are adapted to communicate with a remote system, in order to facilitate one or more of the steps.

16. A method according to claim 9, wherein the test unit is adapted to test a sample from medical purposes, the sample being selected from one or more of blood, saliva, sputum, mucus, serum, plasma, or other bodily fluids.

17. (canceled)

18. (canceled)
